(19) United States
(12) Patent Application Publication
(10) Pub. No.: US 2021/0346446 A1
(43) Pub. Date: Nov. 11, 2021

(54) WATER SOLUBLE CANNABINOID BEVERAGE COMPOSITION

(71) Applicant: HAI BEVERAGES INC., Vancouver (CA)

(72) Inventors: Robert August, Vancouver (CA); Mike McCune, Vancouver (CA); Sazzad Hossain, Richmond (CA)

(73) Assignee: HAI BEVERAGES INC., Vancouver, BC (CA)

(21) Appl. No.: 17/277,790

(22) PCT Filed: Sep. 20, 2019
(86) PCT No.: PCT/CA2019/051352

§ 371 Date: Mar. 19, 2021

Related U.S. Application Data
(60) Provisional application No. 62/734,381, filed on Sep. 21, 2018.

(51) Int. Cl. A61K 36/185; A61K 47/26; A61K 47/10; A61K 9/00; A23L 2/52; A23L 33/105; A23L 2/44; A23L 2/385

(52) U.S. Cl. CPC A61K 36/185; A61K 47/26; A61K 47/10; A61K 9/0095; A23V 2002/00; A23L 33/105; A23L 2/44; A23L 2/385; A23L 2/52

(57) ABSTRACT

A beverage syrup containing cannabinoids can be prepared by solubilizing insoluble cannabinoids in a diluent having dispersion surfactant properties. The diluent can be prepared by mixing a solvent having surfactant properties, such as polysorbate 80, with a co-surfactant, such as propylene glycol. Solid cannabinoids can solubilized by heating the solid concentrate to liquefying it, and then adding the liquid concentrate to the prepared diluent. The resulting syrup can be stored for use in the manufacture of beverages.

### FIELD

Embodiments of this invention relate to a composition for use in a beverage containing water soluble cannabinoids, such as delta-9-tetrahydrocannabinol and/or cannabidiols, and methods of manufacturing the same.

### BACKGROUND

Fountain drinks, or drinks dispensed from a fountain or machine, can be found ubiquitously in restaurants, concession stands, gas stations, malls, and convenience stores. The fountains combine a flavored syrup or syrup concentrate with chilled and/or purified water to make the fountain drinks and be dispensed into beverage containers, typically individual cups. The syrup concentrates are often pumped from a container commonly known as a bag-in-box, which are also known as poly bags. Fountain drinks are a relatively simple and cost efficient way of providing a beverage to individual consumers.

For beverages that are bottled, the manufacturing process is similar, in that a beverage concentrate is manufactured and then stored for later use. The concentrate, usually in liquid form, is then diluted in an aqueous solution or diluent to create the final beverage that is bottled (or canned or otherwise stored in portable containers) for later consumption. During the bottling process, in order to increase shelf life of the beverages, the beverages must be pasteurize before storage and transportation. Any beverage syrup must be able to withstand pasteurization and still maintain its flavor, its taste profile and, for any adult beverages, any desired effects.

The Cannabis sativa plant is plant species comprising Cannabis sativa, Cannabis indica, and Cannabis ruderalis, and is commonly used to produce hemp fiber, hemp oils, and is commonly used to produce tetrahydrocannabinol (THC) for recreational drug use, and cannabidiol (“CBD”) for medicinal purposes. While not completely supported by clinical trials, pharmaceutical compositions containing THC and/or CBD (together commonly referred to as cannabinoids) have been developed to treat a variety of ailments, including treatments for nausea, vomiting, increasing appetite and treatment of chronic pain and muscle spasms.

With the recent trend towards the legalization of Cannabis for medicinal and, in certain cases, recreational use, a complete industry surrounding the cultivation of Cannabis, the sale of Cannabis, and more specifically, pharmaceutical compositions specifically containing cannabinoids have been developed for a range of uses.

Pharmaceutical compositions include oils, pills for oral ingestion, intravenous injections, and eye droplets. To manufacture pharmaceutical compositions containing cannabinoids, cannabinoid concentrates, or extracts are isolated from Cannabis plant materials using various isolation techniques, including fractional distillation. The concentrates, once purified, can be made into a pharmaceutical composition suitable for consumption by patients by the addition of various chemical constituents. Cannabinoids are chemically insoluble in water but can be dissolved in an organic solvent.

In US published patent application 2008/0112895 to Kottayil et al, an aqueous solution of cannabinoids was manufactured by dissolving the cannabinoids in a buffer solution comprising a mixture of a solvent and a co-solvent. In a preferred embodiment, the solvent/co-solvent mixture used was an ethanol/propylene glycol mixture.

However, pharmaceutical compositions, particularly those taken orally or via inhalation have relatively low bioavailability. That is, uptake of the cannabinoids into the body is relatively slow and the desired effects of the cannabinoids are often delayed. Thus, an object of the embodiments of the present invention are related to increasing bioavailability of THC and CBD intake soon after consumption.

In US published patent application 2018/0020699 to Steup a CBD containing beverage was disclosed. More specifically, Steup teaches that a CBD concentrate can be made stable when CBD is combined with at least one emulsifier, such from the group consisting of polysorbates. In a preferred embodiment, the emulsifier is a non-ionic emulsifier from the group consisting of polyethylene glycols.

According to Steup, the CBD containing beverage is preferably produced by introducing the CBD in the form of a stable emulsion (ie. composition of water and at least one emulsifier) into a finished beverage or a corresponding beverage base material, such as a syrup.

Manufacture of water soluble cannabinoids can be achieved, such as in the pharmaceutical industry, by emulsifying cannabinoid concentrates, such as insoluble cannabinoids, in a solvent or by suspending the insoluble cannabinoid in a solvent. However, cannabinoid compositions comprising emulsions and/or encapsulated cannabinoids are not suitable for use in manufacturing adult beverages as the water soluble cannabinoids created by emulsions and/or encapsulated cannabinoids have relatively low bioavailability as they must pass any first pass metabolisms.

As known in the industry, desired effects of a cannabinoid containing beverage ought to be manifested and experienced by the consumer in a relatively short period of time after consumption. This is for several reasons, the most important being for safety reasons, as a large amount of a beverage can be consumed by a person and if the desired effects are not manifested or experienced by the consumer, the consumer may be at risk of consuming too high of a dose of the cannabinoids. Accordingly, there is a need to create a water soluble cannabinoid that has a relatively high bioavailability, such that consumers can experience and feel the effects of cannabinoids in a relatively short period of time after consumption thereof.

### SUMMARY

A syrup containing cannabinoids can be used to manufacture beverages that provide certain desirous effects relatively quickly after consumption of the cannabinoid beverage. To manufacture the syrup, insoluble cannabinoids must be made into a water soluble concentrate for manufacturing beverages thereafter. Insoluble cannabinoids can be solubilized into an aqueous concentrate by creating a suspension conjugate.

In a broad aspect of the present invention, a method for manufacturing a water soluble cannabinoid syrup involves providing a food safe diluent (or water-soluble base solution) having dispersion surfactant properties, providing a liquid concentrate containing cannabinoids, adding the liquid cannabinoid concentrate to the diluent and suspending the cannabinoids in the solution.

### BRIEF DESCRIPTION OF THE DRAWINGS

FIG. 1 is a flow chart illustrating the steps for manufacturing a water soluble cannabinoid syrup;

FIG. 2 is a flow chart illustrating the steps for providing a food safe diluent (or water soluble base solution) having dispersion surfactant properties; and

FIG. 3 is a flow chart illustrating the steps for providing a liquid cannabinoid containing concentrate.

### DESCRIPTION

Embodiments of the present invention generally relate to a method for producing a cannabinoid containing composition that can be readily diluted with water to produce a ready to drink beverage containing cannabinoids. In embodiments, the cannabinoid containing composition or concentrate is in liquid form and stable at room temperature, can be easily stored in storage containers, such as poly bags, and can be easily transported.

As known in the industry, cannabinoids are insoluble in water and cannot simply be mixed with water to create a concentrate beverage syrup for use in the manufacture of adult beverages containing cannabinoids. Accordingly, embodiments of the present invention first require creating a water soluble cannabinoid containing syrup.

The chemistry for making an insoluble product water soluble is well-known and understood. One method is to encapsulate the insoluble product, or otherwise envelope the insoluble product within an envelope or capsule that is water soluble. This creates very small capsules that are water soluble, containing the insoluble product therein. Another method for making an insoluble product water soluble is to create an emulsion using an emulsifier. In such methodologies, the emulsifier assists in creating a dispersion of the insoluble product within the soluble solvent.

As discussed above, the pharmaceutical industries create a water soluble composition containing cannabinoids by creating an emulsion and/or encapsulating cannabinoids with the aid of one or more emulsifiers and/or surfactants. Embodiments of the present invention does not rely on the chemistry of creating an emulsion or an encapsulation, but differs and rather relies on the chemistry of creating a suspension conjugate.

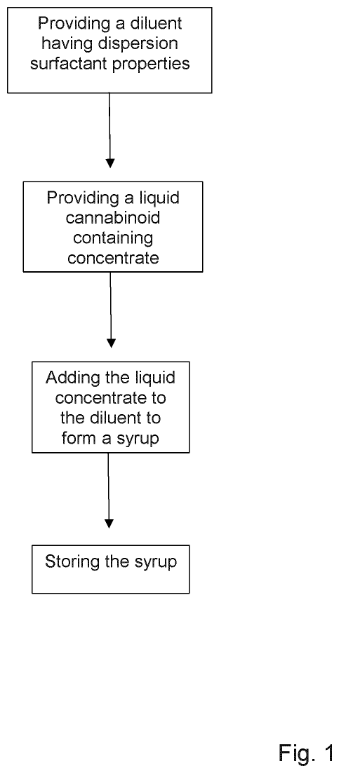

With reference to FIG. 1, in an embodiment, a method for producing a cannabinoid containing composition comprises the steps of initially providing a water soluble base solution (diluent) for suspending a cannabinoid containing concentrate having dispersion surfactant properties. In an embodiment, the diluent can be a food safe solvent having emulsifying and surfactant characteristics for dispersing and suspending insoluble cannabinoids in an aqueous dispersion or suspension. The diluent is then heated to a threshold temperature prior to the addition of a cannabinoid concentrate, cannabinoid isolate or cannabinoid distillate. As the diluent is heated and maintained at the threshold temperature, an appropriate amount of a cannabinoid concentrate is also heated to a threshold temperature to liquefy the concentrate.

In embodiments, solvents that can be used are polysorbate 20, polysorbate 60, polysorbate 80, or any combination thereof.

After the concentrate is liquefied, the liquid concentrate is added to the diluent and mixed together forming a cannabinoid containing syrup. The formed syrup is degassed and allowed to reach equilibrium before it is packaged for storage.

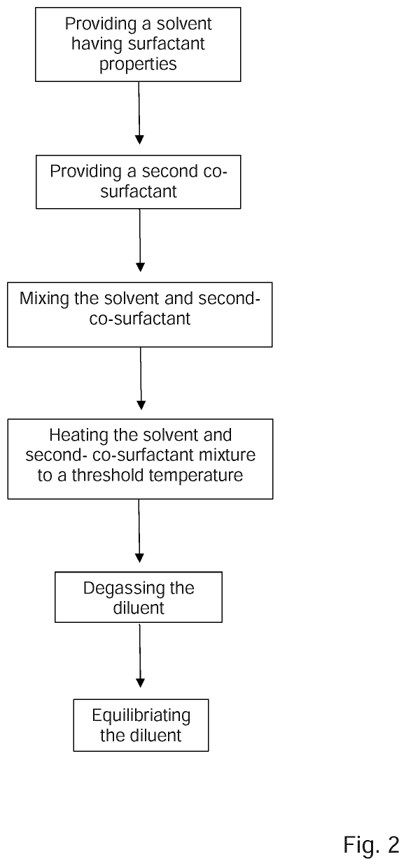

With reference to FIG. 2, in an embodiment, the diluent can comprise the solvent having surfactant properties, and a second co-surfactant, wherein the combination of the surfactant properties of the solvent and the surfactant properties of the co-surfactant synergistically behave to act as a dispersion surfactant for suspending the insoluble cannabinoids within the solvent. In a preferred embodiment, the second co-surfactant further increases the bioavailability of the cannabinoids, and thus obviates the need or requirement for additional constituents that would individually increase bioavailability of cannabinoids. (Discussed in greater detail below).

As shown in FIG. 2, in an embodiment, the diluent can be prepared by placing an appropriate amount of solvent having surfactant properties, such as polysorbate 80, into a mixing tank of appropriate size, and then adding an appropriate amount of the second co-surfactant. As discussed above, in preferred embodiments, the second co-surfactant has properties to act as a buffer and surfactant, but also has properties to increase the bioavailability of the cannabinoids. In an embodiment, the second co-surfactant can be a poly glycol buffer having non-ionic surfactant properties, such as propylene glycol or polyethylene glycol. Applicant notes that polysorbate 80, being a hydrophilic, non-ionic surfactant, an emulsifier, an excipient and a medium length fatty acid chain is suitable for dispersing the cannabinoids therein. In an embodiment, the Applicant suspended 100 mg of cannabinoids per millilitre of the diluent.

In an embodiment, 12 litres of a polysorbate 80 (solvent) can be added to a 20 litre mixing tank, following by 3 litres of propylene glycol (co-surfactant), to create a diluent having a 3:1 (vol:vol) ratio of solvent to co-surfactant. In a preferred embodiment, 10 litres of polysorbate 80 can be mixed with 2 litres of propylene glycol (co-surfactant) in the mixing tank to create a 5:1 ratio diluent. This mixture of solvent and co-surfactant can be heated to a temperature between 70° C. and 110° C. In a preferred embodiment, the mixture containing the solvent and the co-surfactant can be heated to a threshold temperature of about 70° C., and stirred mechanically for about one hour and then allowed to degas and equilibriate for another hour. Once degassing and equilibrium is achieved, the diluent can be stored in appropriate storage containers for later use.

Applicant notes that each of the polysorbate 80 and the propylene glycol individually have surfactant properties (i.e. assists in solubilizing insoluble products in water), but when combined together, the combination of polysorbate 80 and propylene glycol acts as a dispersion surfactant, which creates an even dispersion of an insoluble product, namely, the cannabinoids contained in the cannabinoid concentrate.

In preferred embodiments, the diluent can be enhanced further by the addition of other constituents to improved characteristics. For example, additional bioavailability enhancers, such as vitamin E, and/or preservatives, such as ethylenediamine (EDTA) can be added to improve shelf life, and uptake of the cannabinoids.

In another preferred embodiment, Applicant notes that the co-surfactant propylene glycol is not very palatable, and in order to improve the taste profile of the diluent, polyethylene glycol 400 (PEG-400) can be mixed with the propylene glycol in a volume to volume ration of 1:1.

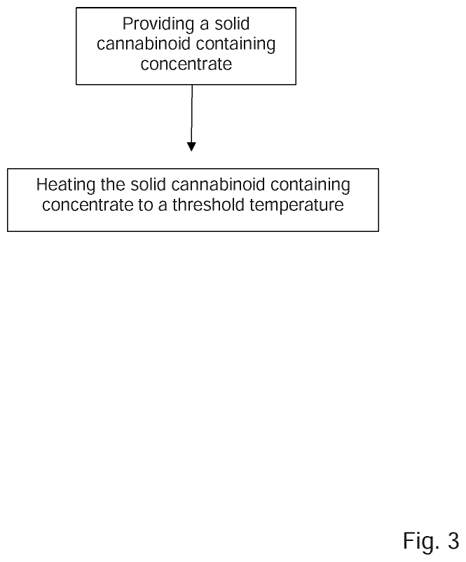

With reference to FIG. 3, and in order to provide a liquid cannabinoid containing concentrate, an appropriate amount of cannabinoid concentrate is placed into an appropriate sized storage container such as a mixing tank. The solid or semi-solid cannabinoid concentrate is heated to a temperature between 70° C. to about 110° C. to liquefy the solid concentrate. In an embodiment, the concentrate can be heated to a threshold temperature of about 80° C., and maintained at the threshold temperature to liquefy the solid cannabinoid concentrate. In a preferred embodiment, 5 kilograms of a solid cannabinoid containing concentrate can be placed into a mixing tank and heated to about the threshold temperature of about 80° C. for about 15 minutes, or until the solid cannabinoid concentrate becomes fluid. Care should be taken as heating the cannabinoid concentrate for too long increases the risk that the desired cannabinoids are converted into a cannabinoid metabolite known as cannabidiol (CBN) having mild psychoactive properties, as well as having sedative properties.

Subsequently, and referring back to FIG. 1, the liquid cannabinoid concentrate is then added to the heated diluent, under constant mixing and is heated to about 80° C. to ensure that the liquid concentrate does not resolidify. The mixture of diluent and cannabinoid concentrate is maintained at about 80° C. (or a temperature sufficiently high enough to prevent the cannabinoid concentrate from resolidifying) and continuously mixed until a suspension conjugate of the diluent and the cannabinoids or cannabinoid syrup is formed. In a preferred embodiment, this mixing process can be between 30 to 45 minutes. After the completion of the cannabinoid syrup, the syrup can be packaged into drums or poly-bags for storage or shipping.

In a preferred embodiment, 5 kilograms of solid cannabinoid concentrate can be liquefied and added to a volume of about 12.5 litres of diluent. However, this ratio of cannabinoid to diluent can be altered to provide various concentrations of cannabinoids available in a final beverage product.

It is commonly known that polysorbate 80 is a medium chain fatty acid and surfactant, and is used in the food industry as an emulsifier, such as in ice cream. It is used globally in the food industry, where it is used as an emulsifier and a solubilizing agent in various food and food products, such as bread, cake mixes, salad dressings, shortening oils and chocolate. Polysorbate 80 is also known to be used as an excipient used to stabilize aqueous formulations of pharmaceutical compositions. It is not known in the industry to use polysorbate 80 as a solvent.

Applicant notes that propylene glycol is a clear, colourless, viscous and synthetic organic compound that is often used in the food industry as a sweetener, but can be used as a solvent in pharmaceuticals. It is also used as an emulsifier in the food industry to assist in mixing together various ingredients and prevent separation of the ingredients. However, in embodiments of the present invention, propylene glycol is used not as a sweetener, but rather to increase the bioavailability of the cannabinoids once the cannabinoid containing beverage is consumed.

Typically. it has been accepted that cannabinoids once consumed will be absorbed into the body within 20 to 60 minutes after consumption, and therefore delaying the onset of the desired effects of cannabinoids. However, consumption of cannabinoids with the propylene glycol increases bioavailability of cannabinoids and decreases the length of time for absorption into the body from the 20 to 60 minutes to about 5 minutes, greatly increasing the efficacy of the cannabinoids.

Polysorbate 80 acts as a surfactant, and forms a liquid, water soluble carrier with propylene glycol (or polyethylene glycol). Cannabinoids, being commonly known for being insoluble (or at least poorly soluble) forms a suspension wherein the individual cannabinoid molecules are dispersed within the polysorbate 80-propylene glycol carrier to the point of being able to withstand heat pasteurization when added to a beverage that requires beings pasteurized.

Applicant is aware that many beverages currently on the market utilize micro-encapsulation to render cannabinoids water soluble. As one skilled in the art would understand, micro-encapsulation is the process in which a target constituent, cannabinoids in this case, is surrounded by a coating to produce many micro capsules. Micro-encapsulation requires the cannabinoids to be digested by the stomach, which delays the onset of the desired effects of cannabinoids and is not able to withstand pasteurization. Once digested, the cannabinoid is then delivered to the liver for first pass metabolism before it is passed to general systemic circulation. Applicant believes that this process takes about 15 to 30 minutes for an average person.

Applicant believes that the suspension created by the 5:1 diluent (vol:vol) of polysorbate 80:propylene glycol bypasses first pass metabolism in the liver, and enables the cannabinoids to enter into general systemic circulation much quicker, decreasing the time for the onset of the desired effects.

1. A method for preparing a water soluble cannabinoid syrup for use in manufacturing beverages comprising the steps of:
providing a food safe diluent having dispersion surfactant properties;
providing a liquid cannabinoid concentrate having cannabinoids therein;
adding the liquid cannabinoid concentrate to the diluent; and
suspending the cannabinoids in the diluent for creating the water soluble cannabinoid syrup,
wherein the food safe diluent increases a bioavailability of the cannabinoids.

2. The method of claim 1, wherein the diluent further comprises a solvent having hydrophilic and non-ionic surfactant properties.

3. The method of claim 2, wherein the solvent further comprises a polysorbate.

4. The method of claim 3, wherein the polysorbate is polysorbate 80.

5. The method of claim 1, wherein the diluent further comprises a co-surfactant.

6. The method of claim 5, wherein the co-surfactant is a poly glycol buffer having non-ionic surfactant properties.

7. The method of claim 6, wherein the poly glycol buffer is propylene glycol.

8. The method of claim 6, wherein the poly glycol buffer further comprises propylene glycol and polyethylene glycol 400.

9. The method of claim 5, wherein the ratio of solvent to co-surfactant is 3:1.

10. The method of claim 9, wherein the ratio of solvent to co-surfactant is 5:1.

11. The method of claim 1, further comprising heating the diluent to a threshold temperature prior to adding the liquid cannabinoid concentrate to the diluent.

12. The method of claim 11, wherein the threshold temperature is in the range of 80° C. to about 110° C.

13. The method of claim 12, wherein the threshold temperate is about 80° C.

14. The method of claim 1, wherein providing a liquid cannabinoid concentrate further comprises heating a solid cannabinoid concentrate to a threshold temperature for liquefying the solid cannabinoid concentrate.

15. The method of claim 14, wherein the threshold temperature is in the range of 70° C. to about 110° C.

16. The method of claim 15, wherein the threshold temperature is about 70° C.

17. The method of claim 1, further comprising adding bioavailability enhancers.

18. The method of claim 1, further comprising adding preservatives.
